(19) United States
(12) Patent Application Publication
(10) Pub. No.: US 2025/0268761 A1
(43) Pub. Date: Aug. 28, 2025

(54) USER-CUSTOMIZABLE BANDAGE

(71) Applicants: Brent Edward Mabey, Salt Lake City, UT (US); Jennifer Bowen, Millcreek, UT (US)

(72) Inventors: Brent Edward Mabey, Salt Lake City, UT (US); Jennifer Bowen, Millcreek, UT (US)

(21) Appl. No.: 19/065,676

(22) Filed: Feb. 27, 2025

Related U.S. Application Data
(60) Provisional application No. 63/558,541, filed on Feb. 27, 2024.

(51) Int. Cl. A61F 13/0246; A61F 13/00

(52) U.S. Cl. CPC A61F 13/0256; A61F 2013/00565; A61F 2013/0071

(57) ABSTRACT

A customizable bandage includes an internal layer, a container layer containing a hardening agent and/or medicinal agent, an external layer surrounding the internal layer and the container layer, an adhesive layer disposed on a lower surface of the external layer, and a padding layer disposed on a region of the lower surface of the external layer. The hardening agent, when disseminated from the container layer, can harden the internal layer into a semi-rigid state.

### CROSS-REFERENCE TO RELATED APPLICATIONS

This application claims priority to and the benefit of U.S. Provisional Patent Application No. 63/558,541, filed on Feb. 27, 2024, the entirety of which is incorporated herein by reference.

### BACKGROUND

### Technical Field

The present disclosure generally relates to wound and injury management. In particular, this disclosure relates to bandages that can be configured to harden into a semi-rigid state following desired placement and user activation, and/or which can contain one or more desired medical agents.

### Related Technology

Wound and injury management has conventionally involved the use of relatively soft bandages such as common adhesive bandages (e.g., Band-Aid®), gauze, and dressing wraps (e.g., ACE® wraps). These types of care products are widely available and easily purchased, and are therefore commonly utilized for at-home use and commonly found in first-aid kits. The prevalence of these types of soft bandages means they are also widely stocked and used in medical practices and hospitals as well.

While these types of soft bandages can be effective for a variety of minor wounds and injuries, broken bones often require the placement of an orthopedic cast to allow the affected bone to be properly set and isolated as it heals. Orthopedic casts were traditionally made by coating a fabric material in calcined gypsum (i.e., Plaster of Paris) and wetting the material to begin the setting process. Modern orthopedic casts usually consist of an inner fabric wrap and an exterior fiberglass wrap impregnated with a polyurethane formulated to cure upon wetting. Orthopedic casts are very rigid and require the use of specialized cutting tools for removal.

As another example, severe lacerations often require the use of surgical suture or “stitches” to sew the wound shut. On some occasions, a tissue glue, usually cyanoacrylate based, can be used along with stitching or as an alternative to stitching for wound closure. While these techniques can be effective, it is not uncommon for the associated wound to be subsequently aggravated or even reopened because of bumping of the wound or strain placed on the wound.

While the above-described types of wound and injury management products have provided many benefits, several limitations remain. The soft and pliable products available for at-home and common first-aid use are often lacking in desired levels of structure and rigidity. For example, someone with a sprained ankle or wrist may wrap the affected joint using a common elastic bandage/dressing. While this can provide compression and limited support to the affected joint, it does not provide much additional orthopedic support. On the other hand, these types of injuries do not justify the use of a full orthopedic cast, which would be overly rigid and could not be easily applied and removed when convenient.

As another example, someone may have a cut that is somewhat too severe to be appropriately closed using standard adhesive bandages, gauze, or dressings. These cuts usually require stitching and its accompanying inconveniences. This can be particularly frustrating for cuts that are not severe, but still too large or deep to be appropriately handled with standard first-aid bandaging.

Accordingly, there is an ongoing need for wound and injury management products capable of addressing issues such as those described above. In particular, there remains a need for bandages capable of providing greater support and structure than the standard adhesive bandages and dressings commonly available in the store and in first-aid kits, but not having the excessive rigidity of orthopedic cast materials. There also remains a need for products that can provide these benefits without the inconveniences and complexities of products that must be applied in a medical setting as with orthopedic casts and stitches.

### SUMMARY

The present disclosure is directed to a customizable bandage to manage or prevent a wound or injury. The disclosed bandages can include one or more agents (e.g., anesthetic, analgesic, antimicrobial, anti-inflammatory, anti-allergy, or combination thereof) that can be selectively released toward target tissue upon user-activation. The one or more agents can include a hardening agent. The customizable bandages described herein can be flexible and pliable when in an uncured position, such that they can be easily manipulated, shaped, and wrapped as desired for placement at a wound or injury site, and then to harden into a “semi-rigid” state after selective release of the hardening agent. Such customizable bandages may be utilized in tissue and/or joint applications.

The customizable bandages disclosed herein may also be referred to as “user-activated” bandages. In embodiments in which the one or more agents include a hardening agent, the bandage may additionally or alternatively be referred to as a “semi-rigid” bandage. While much of the present disclosure relates to bandages that have semi-rigid functionality (i.e., include a hardening agent), it will be understood that the present disclosure also encompasses embodiments that omit a hardening agent and instead include one or more of the other agents disclosed herein. The unmodified term “bandage” may also be used on occasion for simplicity, but the lack of modifiers does not imply difference in scope or functionality.

The one or more agents may include a hardening agent and/or one or more of the other agents disclosed herein. Non-hardening agents included in the one or more agents may be referred to as “medicinal agents.”

In an embodiment, a customizable bandage includes an internal layer, a container layer containing a plurality of agents therein, an external layer surrounding the internal layer and the container layer, an adhesive layer disposed on a lower surface of the external layer, and a padding layer disposed on a region of the lower surface of the external layer. A hardening agent in the plurality of agents, when released from the container layer, hardens the internal layer into a semi-rigid state.

In an embodiment, a method of managing or preventing a wound or injury includes providing the user-activated semi-rigid bandage, applying the semi-rigid bandage to a target site, activating the container layer by releasing at least a portion of the contained agents (e.g., by rupturing, pushing, and/or otherwise disrupting the container layer to allow release of its contents), and allowing a hardening agent of the contained agents to pass into the internal layer of the customizable bandage and cause the internal layer to harden into a semi rigid state.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the aspects, taken in conjunction with the accompanying drawings and the appended claims, all of which form a part of this specification. In the Figures, like reference numerals refer to like components, and the description related to any described component (e.g., external layer 120) is also applicable to corresponding components in other embodiments (e.g., external layer 220 and external layer 320) unless clearly indicated otherwise. The various elements depicted are not necessarily drawn to scale.

FIG. 1A illustrates a plan view of a customizable bandage according to aspects of the present disclosure.

FIG. 1B illustrates a cross-sectional view of a customizable bandage according to aspects of the present disclosure.

FIG. 1C illustrates a bottom view of a customizable bandage according to aspects of the present disclosure.

FIG. 2A illustrates a plan view of a customizable bandage according to aspects of the present disclosure.

FIG. 2B illustrates a cross-sectional view of a customizable bandage according to aspects of the present disclosure.

FIG. 2C illustrates a bottom view of a customizable bandage according to aspects of the present disclosure.

FIG. 3A illustrates a plan view of a customizable bandage according to aspects of the present disclosure.

FIG. 3B illustrates a cross-sectional view of a customizable bandage according to aspects of the present disclosure.

FIG. 3C illustrates a bottom view of a customizable bandage according to aspects of the present disclosure.

FIG. 4A illustrates a plan view of a customizable bandage according to aspects of the present disclosure.

FIG. 4B illustrates a cross-sectional view of a customizable bandage according to aspects of the present disclosure.

FIG. 4C illustrates a bottom view of a customizable bandage according to aspects of the present disclosure.

FIG. 5 illustrates a flowchart of a method for applying a customizable bandage according to aspects of the present disclosure.

### DETAILED DESCRIPTION

### Overview of Semi-Rigid Bandages

The present disclosure relates to customizable bandages, which may be designed to be flexible and pliable when in an uncured position, such that they can be easily manipulated, shaped, and wrapped as desired for placement at a wound or injury site, and then to harden into a “semi-rigid” state after selective release of a hardening agent. The customizable bandages can additionally or alternatively include one or more medicinal agents, such as: an anesthetic (e.g., lidocaine, benzocaine, pramocaine, dibucaine, phenol); an antimicrobial such as an antibiotic (e.g., mupirocin, neomycin, bacitracin, polymyxin B), antifungal (e.g., clotrimazole, miconazole, terbinafine, nystatin), and/or antiviral (e.g., acyclovir, docosanol); an anti-inflammatory (e.g., diclofenac, ibuprofen, ketoprofen, capsaicin, menthol); an anti-allergy agent (e.g., diphenhydramine, doxepin, hydrocortisone, calamine) including combinations of any subset of the foregoing.

As explained in greater detail below, the term “semi-rigid” is used herein to describe a level of rigidity greater than that of standard adhesive bandages or dressings, but less than that of conventional fiberglass orthopedic casts. For example, semi-rigid bandages are designed to be rigid and hard enough that the user cannot simply tear or unwrap them, but soft enough to be cut with handheld scissors (e.g., general household or medical specialty scissors) without great difficulty.

The customizable bandages described herein fill a long-felt need in the art of wound and injury management products. The customizable bandages that incorporate semi-rigid functionality can provide greater support, structure, and stability than standard household and first-aid bandage materials but are less rigid and easier to use than orthopedic casts. Such bandages therefore allow for treatment of wounds and injuries that are not well served by standard bandage materials, but that are not so severe that they require casting. The customizable bandages described herein may be utilized for community/household injury management or prevention and for professional (e.g., doctor's office, emergency room) injury management and prevention. The customizable bandages can release the contained agent(s) and/or may harden into a semi-rigid state based on user's activation.

The semi-rigid bandages are easily removable compared to the conventional fiberglass orthopedic casts. Thus, users and medical professionals may be able to access and assess the wounds and injuries with ease and provide proper hygiene, while proper hygiene with conventional casts cannot be provided without removing the casts and access and assessment of the wounds and injuries cannot be made without removing the casts.

Further, since the semi-rigidity of the semi-rigid bandage may be induced by user's activation, users may be able to change placement of the semi-rigid bandage after noticing that the semi-rigid bandage is placed at an improper position or orientation, and then activate the semi-rigidity after confirming that the semi-rigid bandage is placed at the proper position and orientation.

The one or more medical agents can be disseminated into a sterile padding of the user-activated semi-rigid bandage for deliver to wounds or injuries.

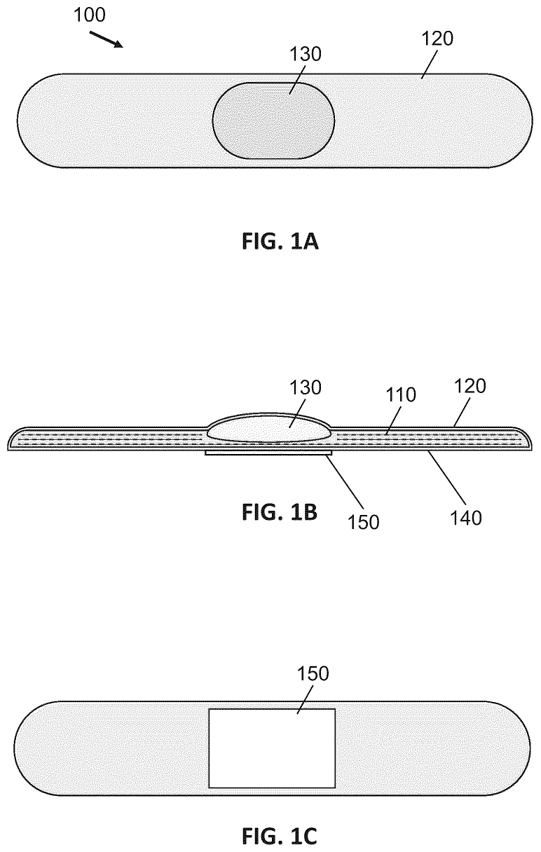

FIG. 1A illustrates a plan view of a customizable bandage 100, FIG. 1B illustrates a cross-sectional side view of the customizable bandage 100, and FIG. 1C illustrates a bottom view of the customizable bandage 100, according to various aspects of the present disclosure. The customizable bandage 100 may include or be provided in the form of a gauze, fabric, mesh, or the like. The customizable bandage 100 may include a self-adhering material such as Coban™. Any suitable soft bandage or combination thereof, as known in the art may be utilized in the customizable bandage 100. The customizable bandage 100 may include multiple layers, including an internal layer 110, an external layer 120, a container layer 130, an adhesive layer 140, and a padding layer 150.

In various aspects, the customizable bandage 100 may comprise elastomeric materials to increase the overall area of the customizable bandage 100, such as common to many dressing wraps.

The internal layer 110 may include any suitable material known in the art capable of hardening to a semi-rigid state upon exposure to a hardening agent contained in the container layer 130. As a result, the customizable bandage 100 can selectively be transformed into a semi-rigid state. The internal layer 110 can additionally or alternatively be configured to disseminate one or more other agents as disclosed herein.

In some embodiments, the internal layer 110 may include a prepolymer (e.g., of polyurethane and/or other polymer), or thermoset polymers capable of polymerizing in the presence of an activator (which may be included in the hardening agent). Thermoset polymers include cyanoacrylate esters and epoxy resins with silane group termini, such as trialkoxy-or trihalo-silane group termini. The hardening agent may include one or more of water, air, catalysts, stabilizers, and/or other components that can initiate hardening/curing of the internal layer 110. In such embodiments, the internal layer 110 may be shielded from air and water (e.g., by the external layer 120) to prevent premature hardening/curing.

Additionally, or alternatively, the internal layer 110 may include a material capable of wicking and/or absorbing the hardening agent upon disruption of the container layer 130. Example materials include cotton, polyester, nylon, microfiber materials, cellulose-based materials, capillary matting, felt, hydrophilic polymers, fibrous materials, sponges/foams, woven materials, and combinations thereof. The hardening agent may include a prepolymer (e.g., any of those disclosed herein) or thermoset polymer (e.g., any of those disclosed herein) capable of polymerizing in the presence of an activator such as air and/or water. For example, upon disruption of the container layer 130, the hardening agent may be distributed substantially throughout the internal layer 110 where it is exposed to air and/or water to initiate hardening/curing.

FIG. 1B, includes dotted lines to indicate dissemination of the hardening agent (and/or other agents) from the container layer 130 through the internal layer 110. After the customizable bandage 100 is placed at a desired position and orientation on a subject, the user can activate the container layer 130 so that the contents of the container layer 130 flow through the internal layer 110. Where the container layer 130 contains a hardening agent, release of the hardening agent can initiate hardening/curing of the internal layer 110.

Activation of the container layer 130 can refer to any manipulation of the container layer 130 that moves at least a portion of the contents of the container layer 130 outside of the container layer 130 and at least partially into the internal layer 110. For example, the container layer 130 can include a shell (e.g., a polymer material) that readily ruptures when exposed to manually applied pressure (e.g., from a user's fingers and/or thumb). The polymer material can be configured with mechanical properties (e.g., material type, thickness) that provide sufficient structural integrity to the container layer yet also enable the seal to be readily broken when intended. In some embodiments, the shell can include a frangible portion (referred to herein as a “crease”) that is intentionally designed to be structurally weaker than the rest of the shell. The crease can be configured to open/disrupt when pressure is applied to the container layer 130. While applying pressure is one method to activate the container layer 130, other methods can additionally or alternatively be used, such as including pull tabs, tear-away sections, push pins, and/or other means for disrupting the container layer 130. In some embodiments, the external layer 120 can form part of the shell. For example, the upper portion of the shell can merge with the coincident portion of the external layer 120.

When the internal layer 110 is hardened, the level of hardness is less than the hardness of a conventional cast and greater than the hardness of a conventional bandage. In other words, the user-activated semi-rigid bandage 100 becomes semi-rigid so that access to and assessment of the wounded lesion can be made easier and proper hygiene can be made.

As an example, the semi-rigid bandage material 100, when cured, may have a Shore A hardness of about 40 to about 100, or about 50 to about 90, or about 60 to about 80. As another example, the user-activated semi-rigid bandage 100, when cured, may have a Young's modulus of about 0.5 to about 3.5 GPa, or about 1 to 3 GPa, or about 1.5 to 2.5 GPa. In functional terms, the user-activated semi-rigid bandage 100, when cured, has a hardness and/or Young's modulus between a conventional dressing wrap (e.g., ACE® wrap) and a conventional orthopedic cast. The foregoing ranges can beneficially meet this functional requirement and are presently preferred. However, other embodiments may have hardness and/or Young's modulus values that lie outside these ranges yet still meet the functional requirement of being between a conventional dressing wrap and a conventional orthopedic cast.

The external layer 120 can be air and/or waterproof to prevent passage of air and/or water into the internal layer 110 region of the customizable bandage 100. Specifically, the external layer 120 may surround the internal layer 110 and the container layer 130, thereby preventing ambient air and/or water from passing into the internal layer 110. Thus, the customizable bandage 100 can maintain its non-rigid or soft state until the user disrupts the container layer 130.

Alternatively, in embodiments where the internal layer 110 does not include components that harden/cure upon exposure to air or water, the external layer 120 can function to provide additional structure and protection to the customizable bandage 100. For example, in embodiments where the container layer 130 includes components that are activatable by air and/or water, and the internal layer 110 includes a wicking and/or absorbent material, the external layer 120 need not necessarily be water and air proof.

The container layer 130 may be located around the center with respect to the horizontal direction and in the vertical direction within the external layer 120. That is, the container layer 130 can be disposed to intersect a central longitudinal axis and/or a central medial/transverse axis defined by the external layer 120.

Agents contained in the container layer 130 may include the hardening agent. The container layer 130 may additionally or alternatively include agents such as analgesics, anesthetics (e.g., lidocaine), clotting factors, and/or antimicrobial compositions. Antimicrobial compositions may include silver, iodine, chlorhexidine, bacitracin, polymyxin B, organic acids such as acetic acid, polyhexamethyl-biguanide (PHMB), permanganate salts, other suitable antiseptics, other antimicrobial formulations, any of the other agents disclosed herein, and combinations thereof.

The container layer 130 may be configured so that the hardening agent and the other agents are contained in separate sections of the container layer 130. In this regard, the container layer 130 may have a first section for the hardening agent and a second section for optional other agents, and the first and second sections can be separated within the container layer 130. For example, the first section may be located at an upper and/or peripheral section of the container layer 130 while the second section may be located in a lower and/or radially inward section of the container layer 130. In such a configuration, when the container layer 130 is activated, the first section may release hardening agent toward both sides (along the longitudinal direction) to deliver hardening agent into the internal layer 110. The optional other agents may flow from the second section toward the bottom of the external layer 120 such as toward the padding layer 150. Alternatively, the hardening agent and any other agents may be included together in the same section, so long as they are sufficiently compatible with each other.

The bottom surface of the external layer 120 can be coated by the adhesive layer 140. Thus, when the adhesive layer 140 is placed on the wounded or injured anatomy, the customizable bandage 100 can adhere thereto. The adhesive layer 140 can be formulated to provide adhesion for the duration of use of the customizable bandage 100, or for a shorter period such as temporarily adhering until the bandage can harden to the semi-rigid state. The entire bottom surface of the external layer 120 may be covered by the adhesive layer 140.

The padding layer 150 may also be placed on the bottom surface of the external layer 120. For example, the padding layer 150 may be positioned opposite the container layer 130. The padding layer 150 may be made of cotton, polyester, rayon, and blends thereof, for example. The shape of the padding layer 150 may have a rectangular shape or any other suitable shape to cover a target wound or injured portion. When the user activates the container layer 130, at least a portion of the agents contained in the container layer 130 may be disseminated into the padding layer 150. Accordingly, the container layer 130 and the padding layer 150 may be in contact, or at least in sufficient proximity, to allow agents released from the container layer 130 to reach the padding layer 150. For example, the bottom surface of the external layer 120 may include an aperture to avoid extending between the container layer 130 and the padding layer 150. Agents reaching the padding layer 150 can thus be administered to the underlying anatomy. Alternatively, or additionally, such agents may be pre-loaded in the padding layer 150.

The padding layer 150 may have a thickness, for example, of about 1/16^{th} of an inch to about ½ of an inch, or preferably about ⅛^{th} of an inch to about ⅜^{th} of an inch. The thickness may be set to be at least as much as a blade height of a set of scissors or other cutting tools used for cutting the semi-rigid bandage. This allows the user to readily place the blade between the semi-rigid bandage 100 in the semi-rigid state and the skin surface when the user desires to remove the semi-rigid bandage 100. That is, the blade height will be substantially equal to or less than the space between the skin surface and the bottom surface of the external layer 120.

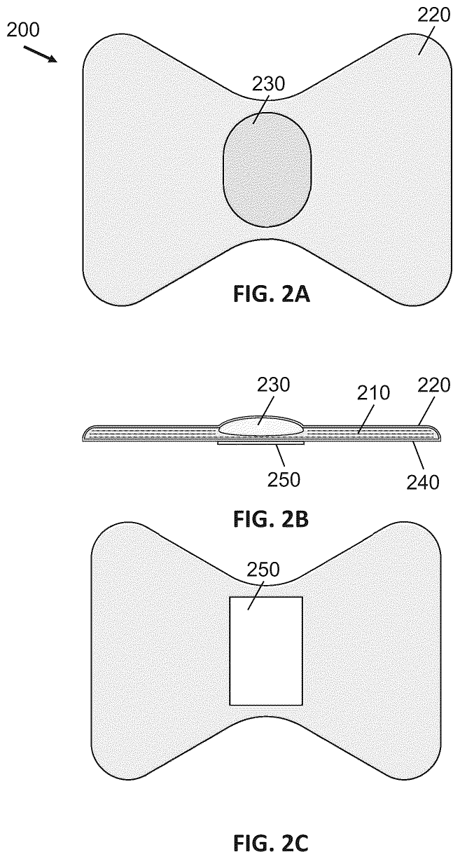

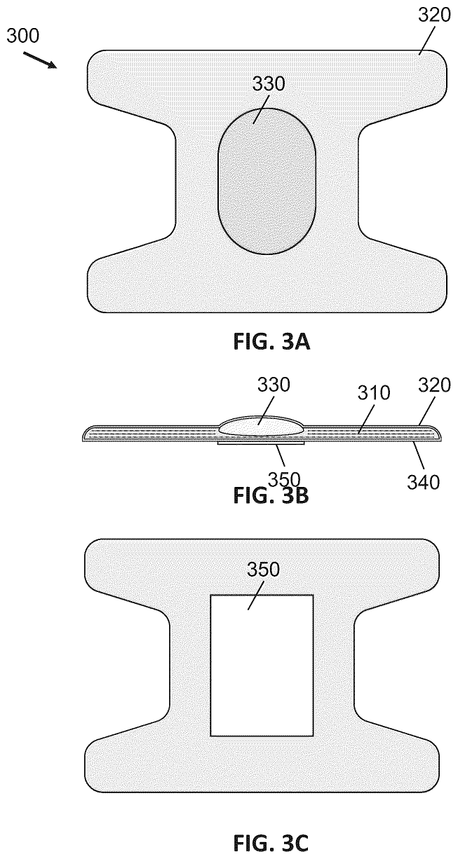

The customizable bandage 100 may have a rectangular shape such as illustrated in FIGS. 1A-1C. Alternatively, a semi-rigid bandage may have different shapes, for example, a butterfly-shaped customizable bandage 200 as illustrated FIGS. 2A-2C or a wedge-shaped customizable bandage 300 as illustrated FIGS. 3A-3C.

The butterfly-shaped customizable bandage 200 has two wings on both ends along the longitudinal or horizontal direction and a narrower center. When the butterfly-shaped customizable bandage 200 becomes semi-rigid, both longitudinal ends can have higher rigidity than the center because of the greater length of the ends relative to the center. In this regard, the butterfly-shaped customizable bandage 200 may be applied to a bending portion of the anatomy (e.g., elbows, knees, finger or toe joints, any other movable joints). The bending portion can bend comparatively easily while the butterfly-shaped customizable bandage 200 maintains its semi-rigid state.

Similarly, the wedge-shaped customizable bandage 300 can include wider outer sections and a center portion with a narrower width. Such a configuration can be useful for fingers and/or toes, for example.

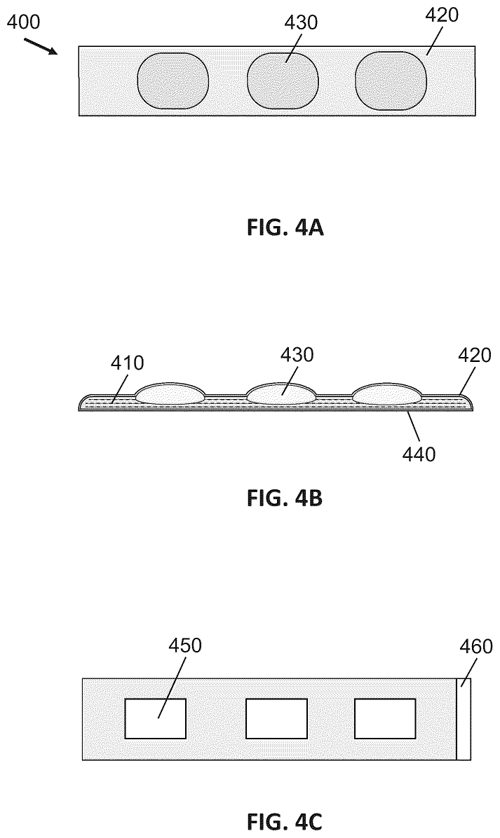

FIGS. 4A-4C show another configuration of a customizable bandage 400. The customizable bandage 400 includes three container layers 430 disposed along the longitudinal direction. Such a configuration can be utilized to cover a relatively long injury, such as a relatively long lesion. The number of container layers 430 may be two, three, or more than three based on the size of the injury, for example.

The number of padding layers 450 may correspond to the number of the container layers 430. Alternatively, there may be more or fewer padding layers 450 relative to the container layers 430. For example, there may be a single padding layer 450 that is larger than the container layers 430. The customizable bandage 400 may omit padding layers 450.

The customizable bandage 400 may include a hook and loop attachment 460. When an injury is wrapped around by the customizable bandage 400, the hook and loop attachment 460 may be attached to the other end of the customizable bandage 400. Thus, the length of the customizable bandage 400 may be longer than the perimeter of the lesion and the number of the container layers 430 may be adjusted based on the perimeter of the lesion.

In embodiments with multiple container layers 430, when the customizable bandage 400 wraps around the lesion, the user can activate the container layers 430 simultaneously or consecutively within a predetermined period (e.g., 30 seconds, 1 minute, 2 minutes, etc.), which can be less than the activation period, during which the internal layer 410 becomes semi-rigid.

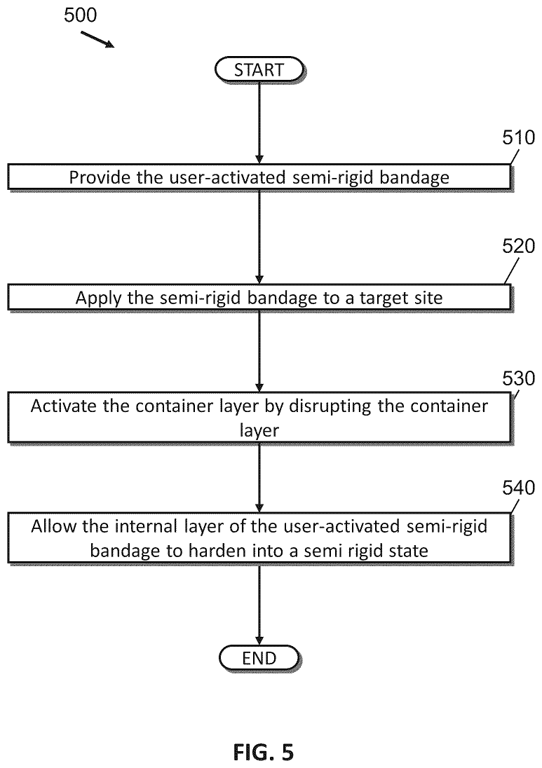

FIG. 5 illustrates a flowchart for a method 500 for managing or preventing a wound or injury according to various aspects of the present disclosure. The method 500 starts by providing a user-activated semi-rigid bandage (i.e., a customizable bandage) at step 510. The initial state of the bandage is relatively soft and flexible.

At step 520, the bandage is applied to a target site. An adhesive layer can assist in adherence to the target site. In a case when the bandage is not applied at a proper position and/or orientation on the lesion, the adhesive layer of the bandage may be easily detached from the target and applied to a proper position and/or orientation on the target.

In an aspect, based on the position of the target site, the shape of the bandage may be selected at step 510. For example, when the lesion is a joint, a star-shaped or wedge-shaped bandage may be provided at step 510 and applied at step 520. When the lesion is large, a bandage such as shown in FIGS. 4A-4C may be provided at step 510 and applied at step 520.

After the customizable bandage has been applied at a proper position and/or orientation on the target site, a user may activate a container layer of the customizable bandage at step 530. The container layer may include a container which contains a hardening agent and optionally other agents (e.g., any agent disclosed herein or combination thereof). When activated, the containers can be disrupted so that the agents are disseminated within the internal layer. In some embodiments containing a hardening agent and one or more other agents, the hardening agent may primarily flow into the internal layer and the one or more other agents may primarily flow into a padding layer.

At step 540, after the internal layer is exposed to the hardening agent after activation, the internal layer of the semi-rigid bandage may be allowed to harden into a semi-rigid state for the activation period.

### Example Aspects

The following clauses represent a non-exhaustive list of example features of the disclosed user-activated semi-rigid bandage:

Clause 1. A customizable bandage comprising: an internal layer; a container layer containing one or more agents, the one or more agents including a hardening agent and/or one or more medicinal agents; an external layer surrounding the internal layer and the container layer; optionally, an adhesive layer disposed on a lower surface of the external layer; and optionally, a padding layer disposed on a lower surface of the external layer, wherein the hardening agent, when included, and internal layer are formulated such that dissemination of the hardening agent from the container layer causes the internal layer to harden into a semi-rigid state.

Clause 2. The customizable bandage of clause 1, wherein the padding layer is longitudinally aligned with the container layer on an opposite side of the internal layer.

Clause 3. The customizable bandage of any preceding clause, wherein the one or more agents comprise an anesthetic, analgesic, antimicrobial, anti-inflammatory, anti-allergy, or combination thereof.

Clause 4. The customizable bandage of any preceding clause, wherein the internal layer comprises a material configured to wick and/or absorb the hardening agent.

Clause 5. The customizable bandage of any preceding clause, wherein the internal layer includes one or more lines through which the hardening agent can flow.

Clause 6. The customizable bandage of any preceding clause, wherein: (i) the hardening agent comprises a curable resin formulated to cure upon exposure to air and/or water; or (ii) the hardening agent comprises water and/or air and the internal layer includes a curable resin formulated to harden upon exposure to the water and/or air.

Clause 7. The customizable bandage of any preceding clause, wherein the external layer is waterproof and/or airproof.

Clause 8. The customizable bandage of any preceding clause, wherein the container layer is configured to be breakable to release the hardening agent upon manually applied pressure.

Clause 9. The customizable bandage of any preceding clause, wherein the user-activated semi-rigid bandage has a rectangular, butterfly, star, spherical, ellipsoid, or wedge shape.

Clause 10. The customizable bandage of any preceding clause, wherein the container layer includes two or more separate sections.

Clause 11. The customizable bandage of clause 10, wherein a first section includes the hardening agent, and a second section includes one or more other agents.

Clause 12. The customizable bandage of any preceding clause, comprising two or more container layers.

Clause 13. The customizable bandage of clause 12, wherein the padding layer comprises two or more pads, each longitudinally aligned with a corresponding container layer.

Clause 14. The customizable bandage of any preceding clause, further comprising a hook and loop attachment.

Clause 15. The customizable bandage of any preceding clause, wherein the container layer includes a crease.

Clause 16. The customizable bandage of clause 15, wherein the crease is configured to break upon exposure to sufficient manually applied pressure to the container layer.

Clause 17. The customizable bandage of clause 16, wherein the container layer is configured to, upon breaking, disseminate the hardening agent through the crease to the internal layer.

Clause 18. A method of managing or preventing a wound or injury, the method comprising: providing the customizable bandage as in any preceding clause; applying the bandage to a target site; activating the container layer to disseminate the hardening agent into the internal layer to cause the internal layer of the bandage to harden into a semi-rigid state.

While many of the examples discussed herein relate to application of a semi-rigid bandage to a “lesion,” it will be understood that the semi-rigid bandage can be applied to various other types of non-lesion injuries, including sprains, bruises, hairline fractures, and the like.

The term “manually applied pressure” refers to pressure that a typical adult user can readily apply using his/her finger or thumb. As an example, a container layer can be configured to break to release the hardening agent when exposed to a threshold pressure of about 2.5 to 10 pounds per square inch (psi), though container layers can be designed to break under less pressure or greater pressure according to particular application needs. The skilled person is capable of testing breaking threshold pressures for a particular container layer design.

While certain aspects of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described aspect, any of the possible alternatives listed for that element or component may generally be used individually or in combination with one another, unless implicitly or explicitly stated otherwise.

When the terms “about,” “approximately,” “substantially,” or the like are used in conjunction with a stated amount, value, or condition, it may be taken to mean an amount, value or condition that deviates by less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an aspect referencing a singular referent (e.g., “widget”) may also include two or more such referents.

It will also be appreciated that embodiments/aspects described herein may include features described in other embodiments/aspects described herein. Accordingly, the various features of a given embodiment/aspect can be combined with and/or incorporated into other embodiments/aspects of the present disclosure. Thus, disclosure of certain features relative to a specific embodiment/aspect of the present disclosure should not be construed as limiting application or inclusion of said features to the specific embodiment/aspect. Rather, it will be appreciated that other embodiments/aspects can also include such features.

1. A customizable bandage comprising:
an internal layer;
a container layer containing one or more agents, the one or more agents comprising a hardening agent;
an external layer surrounding the internal layer and the container layer;
optionally, an adhesive layer disposed on a lower surface of the external layer; and
optionally, a padding layer disposed on a lower surface of the external layer,
wherein the hardening agent and internal layer are formulated such that dissemination of the hardening agent from the container layer causes the internal layer to harden into a semi-rigid state.

2. The customizable bandage of claim 1, wherein the padding layer is longitudinally aligned with the container layer on an opposite side of the internal layer.

3. The customizable bandage of claim 1, further comprising an additional agent comprising an antibiotic, lidocaine, medication, or combination thereof.

4. The customizable bandage of claim 1, wherein the internal layer comprises a material configured to wick and/or absorb the hardening agent.

5. The customizable bandage of claim 1, wherein the internal layer includes one or more lines through which the hardening agent can flow.

6. The customizable bandage of claim 1, wherein: (i) the hardening agent comprises a curable resin formulated to cure upon exposure to air and/or water; or (ii) the hardening agent comprises water and/or air and the internal layer includes a curable resin formulated to harden upon exposure to the water and/or air.

7. The customizable bandage of claim 1, wherein the external layer is waterproof and/or airproof.

8. The customizable bandage of claim 1, wherein the container layer is configured to be breakable to release the hardening agent upon manually applied pressure.

9. The customizable bandage of claim 1, wherein the customizable bandage has a rectangular, butterfly, star, spherical, ellipsoid, or wedge shape.

10. The customizable bandage of claim 1, wherein the container layer includes two or more separate sections.

11. The customizable bandage of claim 10, wherein a first section includes the hardening agent and a second section includes one or more other agents.

12. The customizable bandage of claim 1, comprising two or more container layers.

13. The customizable bandage of claim 12, wherein the padding layer comprises two or more pads, each longitudinally aligned with a corresponding container layer.

14. The customizable bandage of claim 1, further comprising a hook and loop attachment.

15. The customizable bandage of claim 1, wherein the container layer includes a crease.

16. The customizable bandage of claim 15, wherein the crease is configured to break upon exposure to sufficient manually applied pressure to the container layer.

17. The customizable bandage of claim 16, wherein the container layer is configured to, upon breaking, disseminate the hardening agent through the crease to the internal layer.

18. A method of managing or preventing a wound or injury, the method comprising:
applying the semi-rigid bandage of claim 1 to a target site;
activating the container layer to disseminate the hardening agent into the internal layer; and
allowing the internal layer of the customizable bandage to harden into a semi-rigid state.

19. The method of claim 18, wherein the container layer and/or internal layer further includes an anesthetic, analgesic, antimicrobial, anti-inflammatory, anti-allergy agent, or combination thereof.

20. A customizable bandage comprising:
an internal layer;
a container layer containing one or more agents, the one or more agents comprising a medicinal agent;
an external layer surrounding the internal layer and the container layer;
optionally, an adhesive layer disposed on a lower surface of the external layer; and
optionally, a padding layer disposed on a lower surface of the external layer,
wherein the container layer is configured to be selectively disrupted to cause dissemination of the one or more agents from the container layer into one or more other layers.
